(19) United States
(12) Patent Application Publication
(10) Pub. No.: US 2014/0294913 A1
(43) Pub. Date: Oct. 2, 2014

(54) BIODEGRADABLE BONE FILLERS, MEMBRANES AND SCAFFOLDS CONTAINING COMPOSITE PARTICLES

(71) Applicants: Nesrin Hasirci, Ankara (TR); Eda Ayse Aksoy, Ankara (TR); Umran Aydemir Sezer, Ankara (TR)

(72) Inventors: Nesrin Hasirci, Ankara (TR); Eda Ayse Aksoy, Ankara (TR); Umran Aydemir Sezer, Ankara (TR)

(21) Appl. No.: 13/852,949

(22) Filed: Mar. 28, 2013

(51) Int. Cl. A61K 9/70; A61K 38/43; A61K 38/18; A61K 38/02; A61K 38/22

(52) U.S. Cl. CPC A61K 9/70; A61K 38/02; A61K 38/22; A61K 38/18; A61K 38/43
USPC 424/426; 514/1.1; 514/9.7; 514/7.6; 424/94.1

(57) ABSTRACT

This invention is related to bone fillers, hard tissue supporting films and three dimensional scaffolds that contains composite particle of inorganic compound/water soluble polymer (such as β-TCP/Gelatin), that can lead to bone regeneration and release an antibacterial or bioactive agent at the defect area. The bone regenerative hard tissue supporting films and scaffolds were obtained by addition of antibacterial or bioactive agent loaded composite particles into biodegrable polymer (such as PCL) matrix.

### TECHNICAL FIELD

This invention is related to bone fillers that are composite particles of inorganic compounds and water soluble polymer (such as beta-tricalsium phosphate (β-TCP)/Gelatin), that can lead to bone regeneration and release an antibacterial or bioactive agent at the defect area. The bone regenerative hard tissue supporting films and scaffolds were obtained from biodegradable polymer (such as polycaprolactone PCL) matrix by addition of antibacterial or bioactive agent loaded calcium phosphate/water soluble polymer (β-TCP/Gelatin) composite particles.

### BACKGROUND

Bone tissue engineering is a promising area which can be potential alternative solution that possesses better mechanical and biological properties to the tissue in the healing process compared to the traditional methods used currently. The method of bone tissue engineering could be extremely useful in regenerative orthopedic applications that have high incidences of failure secondary to large bone defects. Powders, 2D films or 3D scaffolds are the different forms of the materials used in bone tissue engineering and the forms differ with respect to their usage in varying body parts. Moreover, bone fillers are materials used as injectable hydrogels, pastes or powder materials. Powder forms are used especially in dental applications and they contain bioactive inorganic compounds which accelerate bone formation. These powder forms of materials are also used as a constituent for injectable bone fillers. Among these materials, microparticles are effective especially in filling bone defects of irregular shapes and sizes, in addition to the ability of sustained release of the loaded bioactive agents [Wu et al., 2010]. However, in order to use loaded microparticles as bone fillers, there are three major issues that need to be provided which are bioactivity, degradability and controllable release ability. The combination of biodegradable polymers and bioceramics seems to be a solution for providing these requirements. In the studies focused on bone filler systems as microparticles; the polymer part of the combination has been constituted by natural polymers such as chitosan [Jayasuriya et al., 2009], alginate [Wu et al., 2010] and gelatin [Sivakumar et al., 2002] or synthetic polymers like PCL [Chen et al., 2011], and PLA [Lin et al., 2008 and Maeda et al., 2006]. The inorganic part which gives bioactivity to the bone filler are calcium containing compounds such as calcium biphosphate (CaHPO₄), alpha tricalcium phosphate, beta tricalcium phosphate, octa calcium phosphate, tetra calcium phosphate, amorphous calcium phosphate, calcium sulfate, calcium carbonate (CaCO₃), hydroxyapatite, monetite, bruchite, calcium silicates, etc. It has been shown that the combination of biodegradable polymers with bioceramics in microparticle system demonstrated better cell proliferation and differentiation in in vitro studies, and better tissue-material interaction in in vivo studies [Jayasuriya et al., 2009 and Chen et al., 2011] and good tissue-material interaction in in vivo studies [Lin et al., 2008]. Incorporation of bioactive agent containing microparticles within a 2D or 3D systems can improve both bioactivity of the biomaterial as well as the controlled release of the drug.

Directed bone regeneration is a treatment applied in jaw bones and around teeth. Bone regeneration is a procedure in which a polymeric membrane is placed over the bone graft site. This membrane further encourages new bone to grow and also prevents the growth of scar tissue in the grafted site. Studies of 2D films and membranes in guided bone tissue regeneration have been increasing in recent years, and they are used especially in dental applications. The membrane blocks the unwanted soft tissue invasion and allows ligament fibers so that enhance the bone ingrowth. Once strong ligament fibers attach to root of the teeth, the membrane is removed. The commercially available membranes are made of polymers, including nondegradable polytetrafluoroethylene (PTFE) and biodegradable polylactide, polyglycolide, polycarbonate and collagen. Although PTFE membranes have been indicated best clinical results, biodegradable polymer based membranes have been studied increasingly in the recent years due to the non-requirement of second surgical procedure to remove the membranes [Yang et al., 2009, Song et al., 2007 and Kuo et al., 2009]. As a result, researches have been focused on the biodegradable membranes in order to prevent the second surgery needed for the removal of membrane. In literature, a number of studies about development of novel membranes have published. In order to improve bioactivity and mechanical properties; addition of bioceramics like β-TCP [Kuo et al., 2009], or calcium carbonate [Fujihara et al., 2005] were suggested as fillers. On the other hand, incorporation of antibiotics is another crucial issue due to the open application area of membranes where microorganisms can attack easily. It was reported that direct addition of antibiotic in polymer matrix resulted in burst release [Chung et al., 1997, Park et al., 2000, Kim et al., 2004, and Wu et al., 2010].

Scaffolds are the 3D constructs of tissue engineering which can be replaced into the defected area and mimic the microstructure of targeted tissue [U.S. Pat. No. 7,022,522]. The requirements of scaffold materials are porosity, biocompatibility, and biodegradability. They should show a similar degradation rate with the growth rate of the targeted tissue, and similar mechanical strength with the implantation region [Hou et al., 2003]. Porosity and pore interconnectivity have the key roles in scaffold construction in order to increase the surface area for initial cell attachment and tissue ingrowth with transportation of nutrients and cell wastes [Guarino et al., 2008]. Development of composite scaffolds by using polymers and inorganic materials can be a desired solution with the combination of strength and toughness as the bone tissue constituents [Ramakrishna et al., 2001]. Bone tissue has a composite structure containing elastic collagen and stiff hydroxyapatite. Therefore studies are focused on composite scaffolds mainly containing a biodegradable polymer and additives which can be various bioceramic fillers used for increasing the mechanical strength of the polymer. PCL is one of the preferable materials used as biodegradable polymer constituent in bone tissue engineering composites. Addition of bioceramics or bioglasses into PCL structure can enhance its mechanical strength. Electrospun PCL and β-TCP nanocomposites as biomaterials where β-TCP behaves as bioactive and stiff agent was studied [Eriksen et al., 2008]. Hydroxyapatite (HAp) is also one of the widely used bioceramics in PCL composites [Marra et al., 1999, Calandrelli et al., 2000, Dunn et al., 2001, Choi et al., 2004, Chen et al., 2005, Heo et al., 2009 and Chuenjitkuntaworn et al., 2010]. Kim et al. developed a composite scaffold composed of PCL and phosphate glass with the incorporation of vancomycin as antibiotic agent. They observed lower burst release and higher drug release rate with the addition of phosphate glass [Kim et al., 2005]. Although there are some numbers of studies on development of drug carrying polymer-ceramic composites, there is no optimum device which satisfies the biological, mechanical and physical properties. Therefore, it is preferable and more effective to have a controlled release system formed by addition of bioactive agent into a crosslinked matrix or loaded into the biomaterial so that the burst release would be decreased.

### SUMMARY OF THE INVENTION

Biodegradable hard tissue implants were developed in various physical forms as particles, films and scaffolds. The multifunctionality of the hard tissue support systems are biodegradable, osteoconductive and can control the release of an antibacterial or bioactive agent. The products include;
Composite bone filler particles that can lead to bone healing and release an antibacterial or bioactive agent at the application area. The antibacterial or bioactive agent loaded β-TCP/Gelatin composite particles were developed.
The bone and hard tissue supporting films were obtained from PCL by addition of bioactive agent loaded β-TCP/Gelatin composite particles.
The bone and hard tissue supporting scaffolds composed of PCL and bioactive agent loaded β-TCP/Gelatin micro particles were obtained without using any porogen.
A biodegradable implant containing composite particle can be processed as particle, film, scaffold, sponge and fiber forms where the product can be porous, fibrous or pattered form prepared with or without porogen addition.

### BRIEF DESCRIPTION OF FIGURES

FIG. 1. The product: composite particulate bone filler

FIG. 2. The product: bone and hard tissue supporting films containing composite particulate bone filler

FIG. 3. The product: bone and hard tissue supporting scaffolds containing composite particulate bone filler

FIG. 4. Microscopic images of composite particulate bone fillers

FIG. 5. Disc diffusion test results of bone regenerative hard tissue supporting film: (a) E. Coli, (b) S. Aureus.

FIG. 6. Scaffold implantation into superior iliac crest: (a) bone defect created (b) implantation of scaffolds in the defect, (c) histological examination of the scaffold.

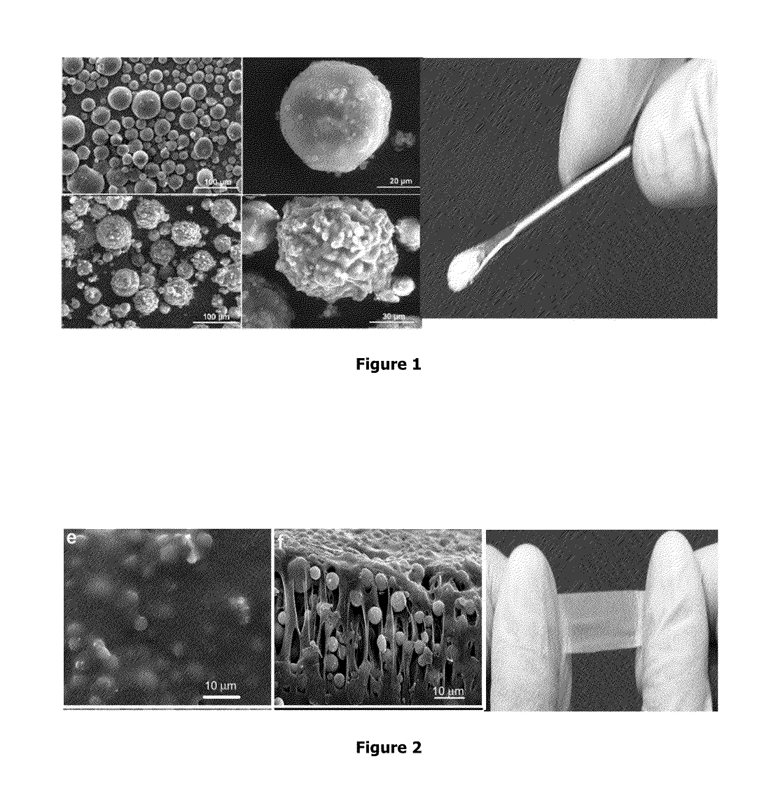

### DETAILED DESCRIPTION OF THE INVENTION

Biodegradable hard tissue implants developed in various physical forms as particulate bone fillers, bone supporting films and tissue scaffolds described in this invention were prepared as the following products and as in the given processing steps.

### Products:

Composite particulate bone fillers
Bioactive agent loaded composite particulate bone fillers
Bone and hard tissue supporting films
Bone and hard tissue supporting scaffolds

### Processing Steps:

### Step 1. Composite Particulate Bone Fillers

Bone fillers are composite particles composed of inorganic compounds and water soluble polymers that can lead to bone regeneration and release an antibacterial or bioactive agent at the bone defect area. The bone regenerative hard tissue supporting films and 3D scaffolds were obtained from biodegradable polymer matrix by addition of antibacterial or bioactive agent loaded calcium phosphate/water soluble polymer composite particles.

Biodegradable water compatible polymer can be natural polymer such as collagen, chitosan, alginate, dextran, gelatin, silk fibroin, hyaluronan, chitin, fibrin, starch, elastin, poly (hydroxy butyrate), cellulose or synthetic polymer such as poly (hydroxyethylmethacrylate), polyethylene glycol, polyethylene glycol methacrylate, polyethylene glycol dimethacrylate, polyethylene glycol diacrylate, poly (ethylene glycol)-poly (ε-caprolactone)-poly (ethylene glycol), poly(vinyl alcohol), polyvinylpyrrolidone, polyimides, polyacrylates, polyurethanes, poly(N-isopropylacrylamide), etc.

Inorganic compound can be alpha-tricalcium phosphate, beta-tricalcium phosphate, hydroxyapatite, monetite, brushite, octacalcium phosphate, tetracalcium phosphate, amorphous calcium phosphate, bioglass, coral, zeolite, silicate.

The ratio of inorganic compounds to water compatible polymer in composite particle material can be 0.001-90%.

Crosslinking agent can be gluteraldehyde, carbodiimide, genipin, phenol/formaldehyde or polyethyleneimine with the ratio of 0.00-50% of composite particle material.

A composite particle material, containing inorganic compound or calcium phosphate and water compatible polymer can be in the particle size range of 10 nanometer-0.5 millimeter. The particle size of composite particle material can be arranged and produced according to bone defect size.

As an example, β-TCP/Gelatin composite particles were prepared with water-in-oil emulsion process. For this purpose, β-TCP powder was put into warm gelatin aqueous solution and was suspended. This suspension was added into oil phase. Glutaraldehyde (GA) solution was added to the medium as crosslinker agent. The mixture was cooled and washed with acetone to remove the oil phase. The solution was filtered and the obtained micro particles were kept at room temperature to dry.

### Step 2. Bioactive Agent Loading to Composite Particulate Bone Fillers

Bioactive agent can be antibacterial agent, protein, drug, hormone, growth factor, antibiotic, antifungal, vitamin, enzyme. The ratio of bioactive agent is 0.00 mg-1000 mg per one gram of composite particle material.

As an example a bioactive agent like an antibiotic as gentamicin was mixed during composite particulate material preparation process or added onto synthesized β-TCP/gelatin microparticles.

### Step 3. Bone and Hard Tissue Supporting Films

The bone and hard tissue supporting films were obtained from biodegradable polymer by addition of bioactive agent loaded composite particles.

Biodegradable polymer can be synthetic polymers such as poly(propylene fumarate), polyglycolides, polylactides, polydioxanone, poly(trimethylene carbonate), polyurethanes, poly(ester amides), poly(ortho esters), polyanhydrides, poly(alkyl cyanoacrylates), polyphosphazenes, polyesters, polycaprolactone and their blends and copolymers or natural polymers such as collagen, gelatin, chitosan, cellulose, fibrin, hyaluronan, dextran, protein, polysaccharides, starch and their blends and copolymers.

The ratio of a composite particles to the biodegradable polymer matrix is 0.0-80 wt %.

As an example composite particulate materials were added in to PCL prepared with solvent casting or melt process. Antibacterial or bioactive agent loaded composite particulate material; at different weight percents were added to the PCL solution, stirred to obtain homogeneous dispersion. The mixtures were molded and then dried.

### Step 4. Bone and Hard Tissue Supporting Scaffolds

The bone and hard tissue supporting scaffolds were obtained from biodegradable polymer by addition of bioactive agent loaded composite particles.

Antibacterial or bioactive agent loaded composite particulate materials with varying ratios were added into PCL solution by stirring. The mixtures were molded in three-dimensional blocks and lyophilized.

### EXAMPLE

### Composite Particulate Material as Bone Fillers

The multifunctional bone filler composites which contain an antibiotic were produced for healing and supporting bone defects. The β-TCP/Gelatin composite systems were prepared in different compositions by changing β-TCP/Gelatin ratios and by using different concentrations of glutaraldehyde (GA) which was used for crosslinking of gelatin. In order to make the system antibacterial, a bioactive agent gentamicin with known amount of was loaded to β-TCP/Gelatin particles.

For this purpose, suspension was prepared by using β-TCP and gelatin aqueous solution and an oily phase. Constant amount of Glutaraldehyde solution (2 mL of 2% solution) was added to the medium as crosslinker agent. The mixture was cooled, washed and filtered via washing with acetone. Table 1 shows the compositions of composite particles. A known amount of gentamicin (0.5 mL, 80 mg/mL) as bioactive agent was loaded to β-TCP and β-TCP/Gelatin particles at room temperature.

TABLE 1
Composition of Composite Particles

| Sample | Composition | β-TCP/Gelatin Ratio (w/w) |
|---|---|---|
| G-2 | Gelatin-2GA | 0.00 |
| 0.25β/G-2 | 0.25 β-TCP/Gelatin-2GA | 0.25 |
| 0.50β/G-2 | 0.50 β-TCP/Gelatin-2GA | 0.50 |
| 1.00β/G-2 | 1.00 β-TCP/Gelatin-2GA | 1.00 |

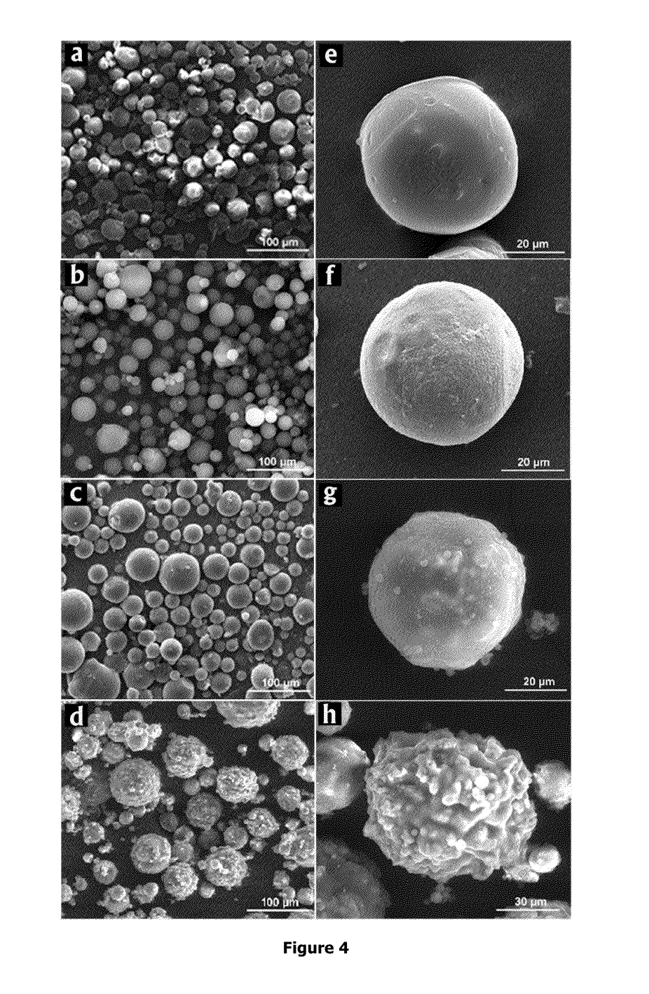

The scanning electron microscope (SEM) images of composite particles prepared with different β-TCP/gelatin ratios are given in FIG. 4. As seen from FIG. 4, with an increase in β-TCP ratio the particle size of composite particles increases and also surface roughness increases. The particle size of these composite particles is important for the bone defect size. The proper particle size (nano (nm) size, micro (μm) size, mili (mm) size) and morphology (sphere, elliptic, block, cubic, cylindrical, random etc.) can be produced with this method so that it could be applicable to the bone defect area. Also, the surface roughness of composite particle is a critical and important condition for cell adhesion and that roughness can lead more efficient cell differentiation in bone regeneration.

### Bone and Hard Tissue Supporting Films

Bone and hard tissue supporting films were produced by addition of gentamicin loaded β-TCP/Gelatin composite particles to PCL. These products can be seen from FIG. 2.

Gentamicin loaded β-TCP/Gelatin composite; at different weight percents (10%, 30% or 50%), were added to the PCL solution, molded and dried.

The mechanical tensile test results of the bone and hard tissue supporting films are given in Table 2. The composite particle ratio changes the mechanical properties of bone and hard tissue supporting films.

TABLE 2
The mechanical tensile test results of the bone and hard tissue
supporting films

| | Ultimate | Youngs | Elongation |
|---|---|---|---|
| | Tensile Strenght | Modulus | at Break |
| Sample | UTS (MPa) | E (MPa) | EAB% |
| PCL | 21 ± 5 | 180 ± 28 | 1439 ± 126 |
| PCL-10β/G | 11 ± 3 | 178 ± 30 | 696 ± 81 |
| PCL-30β/G | 8 ± 1 | 200 ± 37 | 329 ± 77 |
| PCL-50β/G | 6 ± 1 | 240 ± 33 | 192 ± 27 |

### Antibacterial Effect of Bone and Hard Tissue Supporting Films

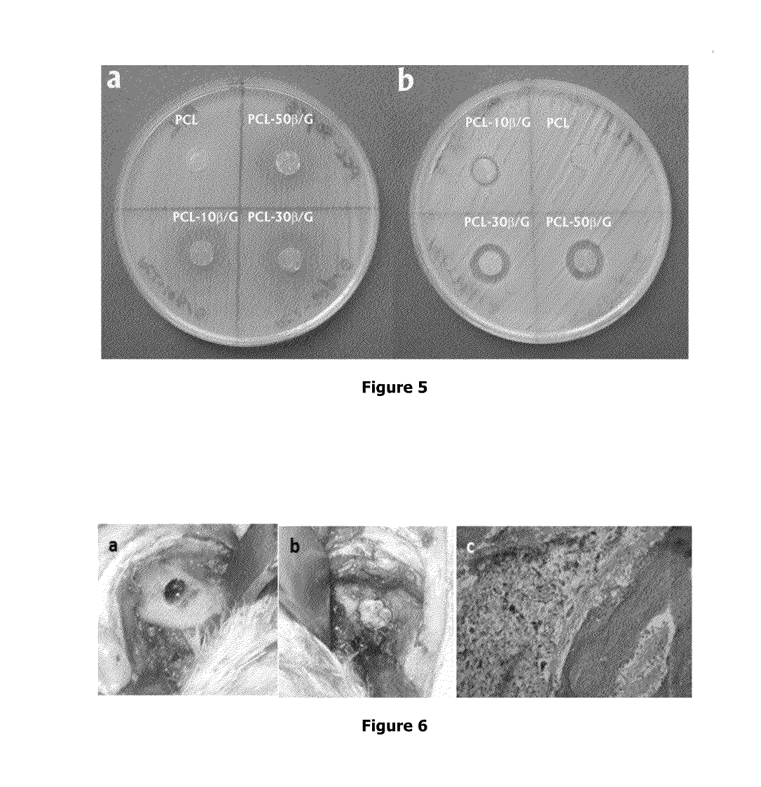

Antibacterial assays against gram negative E. Coli and gram positive S. Aureus were carried out by examining the bacterial growth over 24 h period and results are shown in FIG. 5. Pure PCL material did not indicate any antibacterial activity against both S. Aureus and E. Coli under the test conditions as seen from the absence of zone of inhibition. Bone and hard tissue supporting films containing gentamicin loaded β-TCP/Gelatin composite microparticles had shown an antibacterial affect against S. Aureus and E. Coli. The antibacterial activities of the bone and hard tissue supporting films increased as the ratio of composite particulate material was increased.

### Bone and Hard Tissue Supporting Scaffolds

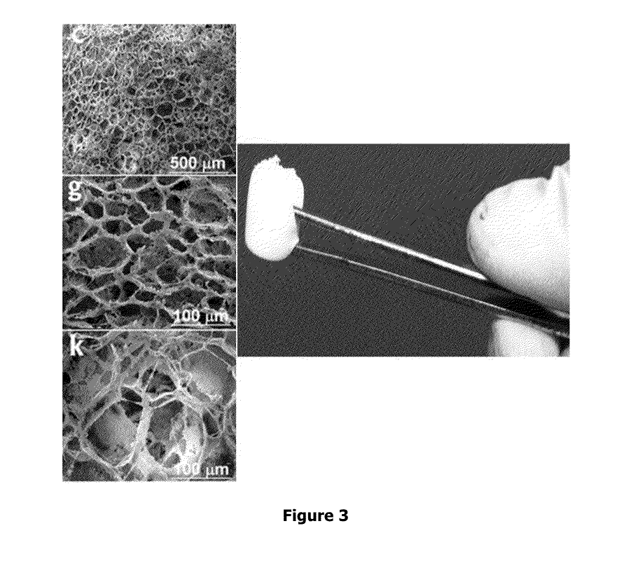

Bone and hard tissue supporting scaffolds were produced by addition of gentamicin loaded β-TCP/Gelatin composite microparticles into PCL solution and lyophilization of the mixture. These products can be seen from FIG. 3.

In vivo Application

The surgical protocols of this study were approved by Cukurova University Animal Research Ethical Committee (Adana, Turkey). Bilateral cylindrical bone defects (Diameter: 5 mm, Height: 4 mm) were created on the iliac crests of rabbits with a pneumatic drill and bone and hard tissue supporting scaffolds were fit-grafted into the defects (FIG. 6). After 8 weeks of implantation, no inflammation was observed on the application area and bone healing occurred by formation tissue regeneration while a decrease in the size of the filler occurred.

### REFERENCES

Calandrelli L., Immirzi B., Malinconico M., Volpe M., Oliva A., Ragione F., ‘Preparation and characterisation of composites based on biodegradable polymers for “in vivo” application’, Polymer., 41: 8027-8033, 2000.
Chen B., Sun K., ‘Poly (ε-caprolactone)/hydroxyapatite composites: effects of particle size, molecular weight distribution and irradiation on interfacial interaction and properties’, Polym Test., 24:64-70, 2005.
Chen J P, Chang Y S. Preparation and characterization of composite nanofibers of polycaprolactone and nanohydroxyapatite for osteogenic differentiation of mesenchymal stem cells. Colloids Surf B Biointerfaces. 86(1):169-75, 2011
Choi D., Marra K., Kumta P., ‘Chemical synthesis of hydroxyapatite/poly(e-caprolactone) composites’, Mater Res Bull., 39:417-432, 2004.
Chuenjitkuntaworn B., Inrung W., Damrongsri D., Mekaapiruk K., Supaphol P., Pavasant P., ‘Polycaprolactone/Hydroxyapatite composite scaffolds: Preparation, characterization, and in vitro and in vivo biological responses of human primary bone cells’, J Biomed Mater Res A., 94:241-51, 2010.
Chung C. P., Kim D. K., Park Y. J., Nam K. H., Lee S.-J., ‘Biological effects of drug-loaded biodegradable membranes for guided bone regeneration’, J Periodontal Res., 32:172-175, 1997.
Dunn A., Campbell P., Marra K., ‘The influence of polymer blend composition on the degradation of polymer/hydroxyapatite biomaterials’, J Mater Sci Mater Med., 12:673-677, 2001.
Erisken C., Kalyon D. M., Wang H., ‘Functionally graded electrospun polycaprolactone and β-tricalcium phosphate nanocomposites for tissue engineering applications’, Biomaterials., 29:4065-4073, 2008.
Fujihara K., Kotaki M., Ramakrishna S., ‘Guided bone regeneration membrane made of polycaprolactone/calcium carbonate composite nano-fibers’, Biomaterials., 26:4139-4147, 2005
Guarino V., Ambrosio L., ‘The synergic effect of polylactide fiber and calcium phosphate particle reinforcement in poly ε-caprolactone-based composite scaffolds’, Acta Biomater., 4:1778-1787, 2008.
Heo S.-J., Kim S.-E., Wei J., Hyun Y.-T., Yun H.-S., Kim D.-H., Shin J. W., Shin J.-W., ‘Fabrication and characterization of novel nano- and micro-HA/PCL composite scaffolds using a modified rapid prototyping process’, J Biomed Mater Res A., 89:108-116, 2009.
Jayasuriya C. A., Bhat A., Rapid biomineralization of chitosan microparticles to apply in bone regeneration. J. Biomed. Mater. Res. A., 93, 1280 (2010).
Johari N., Fath M. H, Golozar M. A., ‘Fabrication, characterization and evaluation of the mechanical properties of poly (e-caprolactone)/nano-fluoridated hydroxyapatite scaffold for bone tissue Engineering Compos Part B-Eng., 2012.
Kim H. W., Knowles J. C., Kim H.-E., ‘Hydroxyapatite and gelatin composite foams processed via novel freeze-drying and crosslinking for use as temporary hard tissue scaffolds’, J Biomed Mater Res A., 72:136-145, 2005.
Kim H. W., Knowles J. C., Kim H. E., ‘Effect of biphasic calcium phosphates on drug release and biological and mechanical properties of poly(ε-caprolactone) composite membranes’, J Biomed Mater Res A., 70:467-479, 2004.
Kim H. W., Yoon B. H., Kim H. E., ‘Microsphere of apatite-gelatin nanocomposite as bone regenerative filler’, J Mater Sci Mater Med., 16:1105-1109, 2005.
Kuo S. M., Chang S. J., Niu G. C. C., Lan C. W., Cheng W. T., Yang C. Z., ‘Guided tissue regeneration with use of β-TCP/chitosan composite membrane’, J Appl Polym Sci., 112:3127-3134, 2009.
Lin F.-H., Yao C.-H., Sun J.-S., Liu H.-C., Huang C.-W., ‘Biological effects and cytotoxicity of the composite composed by tricalcium phosphate and glutaraldehyde cross-linked gelatin’, Biomaterials., 19:905-917, 1998.
Lin X., Bo W., Guang Y., Gauthier M., Poly(Lactic Acid)-Based Biomaterials: Synthesis, Modification and Applications Biomedical Science, Engineering and Technology Biomedical Science, Engineering and Technology Edited by Prof. Dhanjoo N. Ghista, 2008
Maeda H, Kasuga T., ‘Preparation of poly(lactic acid) composite hollow spheres containing calcium carbonates’; Acta Biomater. (4):403-408,2006
Marra K., Szem J., Kumta P., Dimilla P., Weiss L., ‘In vitro analysis of biodegradable polymer blend/hydroxyapatite composites for bone tissue engineering’, J Biomed Mater Res A., 47:324-335, 1999.
Park J. K., Yeom J., Oh E. J., Reddy M., Kim J. Y., Cho D.-W., Lim H. P., Kim N. S., Park S. W., Shin H.-I., Yang D. J., Park K. B., Hahn S. K., ‘Guided bone regeneration by poly(lactic-co-glycolic acid) grafted hyaluronic acid bi-layer films for periodontal barrier applications’, Acta Biomater., 5:3394-3403, 2009.
Park Y. J., Lee Y. M., Park S. N., Lee J. Y., Ku Y., Chung C. P., Lee S. J., ‘Enhanced guided bone regeneration by controlled tatracyline release from poly(L-lactide) barrier mambranes’, J Biomed Mater Res A., 51:391-397, 2000.
Ramakrishna S., Majer J., Wintermantel E., Leong K. W., ‘Biomedical applications of polymer-composite materials: a review’, Compos Sci Technol., 61:1189-224, 2001.
Sivakumar M., Rao K. P., ‘Preparation, characterization and in vitro release of gentamicin from coralline hydroxyapatite-gelatin composite microspheres’, Biomaterials., 23:3175-3181, 2002.
Song J.-H., Kim H.-E., Kim H.-W., ‘Collagen-apatite nanocomposite membranes for guided bone regeneration’, J Biomed Mater Res B., 83:248-257, 2007.
U.S. Pat. No. 7,022,522 ‘Macroporous polymer scafold containing calcium phosphate particles’ 2006.
Wu C., Yufang Z., Chang J., Zhang Y., Xiao Y., ‘Bioactive inorganic-materials/alginate composite microspheres with controllable drug-delivery ability’. J Biomed Mater Res B., 94:32-43, 2010.

I claim:

1. A biodegradable bioactive implant material comprising: biodegradable polymer matrix containing bioactive agent loaded composite particle material of inorganic compound water soluble polymer.

2. A biodegradable bioactive implant material according to claim 1 wherein water soluble polymer is selected from a group consisting of collagen, chitosan, alginate, dextran, gelatin, protein, silk fibroin, hyaluronan, chitin, fibrin, starch, elastin, poly(hydroxy butyrate), cellulose, poly(hydroxyethylmethacrylate), polyethylene glycol, polyethylene glycol methacrylate, polyethylene glycol dimethacrylate, polyethylene glycol diacrylate, poly(ethylene glycol)-poly(ε-caprolactone)-poly(ethylene glycol), poly(vinyl alcohol), polyvinylpyrrolidone, polyimides, polyacrylates, polyurethanes and poly(N-isopropylacrylamide).

3. A biodegradable bioactive implant material according to claim 2, where water soluble polymer is gelatin.

4. A biodegradable bioactive implant material according to claim 1, wherein inorganic compound is selected from a group consisting of beta-tricalcium phosphate, alpha-tricalcium phosphate, hydroxyapatite, monetite, brushite, octacalcium phosphate, tetracalcium phosphate, amorphous calcium phosphate, calcium sulfate, bioglass, coral, zeolite and silicate.

5. A biodegradable bioactive implant material according to claim 4, wherein inorganic compound is beta-tricalcium phosphate

6. A biodegradable bioactive implant material according to claim 1 wherein the ratio of inorganic compound to water soluble polymer in composite particle material is 0.01-90%.

7. A biodegradable bioactive implant material according to claim 1 wherein composite particle material is crosslinked with gluteraldehyde, carbodiimide, diisocyanates, genipin, phenol/formaldehyde or polyethyleneimine, or their di or multi mixtures.

8. A biodegradable bioactive implant according to claim 7 wherein crosslinker ratio is between zero and fifty percent.

9. A biodegradable bioactive implant material according to claim 1 wherein bioactive agent is selected from a group consisting of antibacterial agent, drug, hormone, growth factor, antibiotic, antifungal, vitamin, protein and enzyme.

10. A biodegradable bioactive implant material according to claim 1 where bioactive agent is between 0.00 mg and 1000 mg per one gram of composite particle material.

11. A biodegradable bioactive implant material according to claim 1 where biodegradable polymer matrix is selected from a group consisting of poly(propylene fumarate), polyglycolides, polylactides, polydioxanone, poly(trimethylenecarbonate), polyurethanes, poly(esteramide), poly(ortho esters), polyanhydrides, poly(alkyl cyanoacrylate), polyphosphazenes, polyesters, polycaprolactone, collagen, gelatin, chitosan, cellulose, hyaluronan, dextran and starch.

12. A biodegradable bioactive implant material according to claim 11 where biodegradable polymer matrix is polycaprolactone.

13. A biodegradable bioactive implant material according to claim 1 where particle size of composite particle material is in between 1 nm and 10 mm.

14. A biodegradable bioactive implant material according to claim 1 where the ratio of a composite particle material to biodegradable polymer is between zero and eighty wt percentage.

15. A biodegradable bioactive implant material according to claim 1 processed as particle, film, scaffold, sponge and fiber forms.

16. A biodegradable bioactive implant material according to claim 15 wherein sponge can be porous, fibrous or patterned form prepared with or without porogen addition.
